(19) United States
(12) Patent Application Publication
(10) Pub. No.: US 2022/0079930 A1
(43) Pub. Date: Mar. 17, 2022

(54) Therapeutic Cancer Immune Modulation by Treatment with mTOR Inhibitors

(71) Applicant: Brain Cancer Research Institute, san diego, CA (US)

(72) Inventors: Thomas Ichim, San Diego, CA (US); Santosh Kesari, San Diego, CA (US)

(21) Appl. No.: 17/222,664

(22) Filed: Apr. 5, 2021

Related U.S. Application Data
(60) Provisional application No. 62/970,144, filed on Feb. 4, 2020.

(51) Int. Cl. A61K 31/4462; C07K 16/28; A61P 35/00

(52) U.S. Cl. CPC A61K 31/4462; A61P 35/00; C07K 16/2827; C07K 16/2818

(57) ABSTRACT

Disclosed are means, methods, and protocols useful for treatment of cancer through the previously unknown immune stimulatory effects of mTOR inhibitors and derivatives/analogues thereof. In one embodiment the invention provides the use of mTOR inhibitors to overcome cancer mediated immune exhaustion/anergy. While in conventional situations it is widely known that mTOR inhibitors possesses immune suppressive functions, hence their use in prevention of allograft rejection, our findings suggest that these inhibitors may overcome cancer induced immune suppression and/or exhaustion of immune cell proliferative activities. In some embodiments mTOR inhibitors are utilized together with checkpoint inhibitors. In other embodiments, mTOR inhibitors are administered after lymphodepletion to augment effects of homeostatically expanded lymphocytes.

### CROSS-REFERENCE TO RELATED APPLICATIONS

This application claims priority to U.S. Provisional Application No. 62/970,144, filed Feb. 4, 2020, which is incorporated by reference in its entirety.

### FIELD OF THE INVENTION

The invention pertains to the field of immune modulation, and more specifically the use of mTOR inhibitors to support immune activation, as opposed to immune suppression, such as the for the treatment of cancer

### BACKGROUND

mTor inhibitors have been traditionally used as immune suppressants. The teachings herein utilize mTor inhibitors for immune activation.

### SUMMARY

Various aspects of the invention are enumerated in the following paragraphs. Preferred embodiments are directed to a method of stimulating immune responses in a cancer patient comprising the steps of: a) identifying a patient suffering from cancer possessing a reduced immune response; and b) administering an effective amount of an mTOR inhibitor to augment said immune response.

Said immune response can be associated with T cells acquiring antigen specific and/or antigen non-specific proliferative and/or cytokine producing responses and/or cytotoxic responses. Said immune response can be T cell proliferation in response to a combination of Signal 1 and Signal 2. Signal 1 can be a T cell receptor generated signal and wherein said Signal 2 is a costimulatory molecule generated signal. Said costimulatory molecule can be selected from the group consisting of: a) CD40; b) CD80; c) CD86; d) 4.1bb; e) OX40; f) IL-2; g)) IL-7; h) IL-11; i) IL-12; j) IL-15; k) IL-17; 1) IL-18 and m) interferon gamma. Said immune response can be reduced as a result of a tumor associated immune suppressive factor. Said tumor associated immune suppressive factor can be selected from the group consisting of: a) PGE-2; b) TGF-beta; c) VEGF; d) IL-10; e) PD-1L; f) arginase catabolites; g) indolamine 2,3 deoxygenase catabolites; h) free adenosine; and i) soluble fas ligand trimers. Said mTOR inhibitor can be selected from the group consisting of: a) everolimus; b) sirolimus; c) temsirolimus; d) dactolisib; e) GSK2126458; f) XL765; g) AZD8055; h) INK128/MLN0128; i) OSI0271 and j) RapaLinks.

Certain embodiments herein are directed to methods of treating cancer comprising the steps of: a) administering to a cancer patient a checkpoint inhibitor; and b) administering to said cancer patient an mTOR inhibitor. Further embodiments include methods wherein the checkpoint inhibitor is an agent blocking inhibitory immune cell signaling. Further embodiments include methods wherein the checkpoint inhibitor is Pembrolizumab, Nivolumab, Atezolizumab, or Ipilimumab.

Further embodiments include methods wherein said checkpoint inhibitor blocks an inhibitory receptor selected from the group consisting of: a) a siglec receptor; b) iL-10 receptor; c) IL-13 receptor; d) CTLA-4; e) PD-1; f) PD-1L; and g) TGF-beta receptor. Further embodiments include methods wherein said mTOR inhibitor is selected from a group comprising of: a) everolimus; b) sirolimus; c) temsirolimus; d) dactolisib; e) GSK2126458; f) XL765; g) AZD8055; h) INK128/MLN0128; i) OSI0271 and j) RapaLinks. Further embodiments include methods wherein a stimulator of innate immunity is further added in order to treat said cancer patient. Further embodiments include methods wherein said innate immune stimulator induces activation of dendritic cells and/or natural killer cells. Further embodiments include methods wherein said innate immune stimulator is Poly IC. Further embodiments include methods wherein said innate immune stimulator is beta glucan.

### DETAILED DESCRIPTION OF THE INVENTION

The teachings herein are directed to the use of mTOR inhibitors to support immune activation, which is markedly non-obvious in the context of the classical use of mTOR inhibitors which is for immune suppression. For practice of the invention, numerous mTOR inhibitors may be used, examples of which include everolimus, sirolimus, temsirolimus, dactolisib, GSK2126458, XL765, AZD8055, INK128/MLN0128, OSI027, and RapaLinks.

In one specific aspect of the invention an mTOR inhibitor is administered together with a checkpoint inhibitor. Checkpoint inhibitors may be antibodies, antibody agents, gene silencing means, gene editing means, and small molecules. Numerous immunologically inhibitory molecules exist that function as immunological checkpoints that are useful for the practice of the invention, for example, CTLA4, PD1, PD1-L, IL-10 receptor, TGF-beta receptor, and CD200.

In another embodiment of the invention, mTOR inhibitors are administered together with activators of innate immunity. Said activators are known in the art and include agents that activate toll-like receptors (TLRs), which are pattern recognition receptors which are part of human innate immune system that recognize and mediates early response to tissue injury, followed by activation of the adaptive immune system. Besides these exogenous pathogen-associated molecular patterns (PAMP), TLRs can also bind with damage-associated molecular patterns (DAMP) produced under stress or by tissue damage or cell apoptosis. It is believed that TLRs build a bridge between innate immunity and autoimmunity. There are five adaptors to TLRs including MyD88, TRIF, TIRAP/MAL, TRAM, and SARM. Upon activation, TLRs recruit specific adaptors to initiate the downstream signaling pathways leading to the production of inflammatory cytokines and chemokines. Under certain circumstances, ligation of TLRs drives to aberrant activation and unrestricted inflammatory responses, thereby contributing to the perpetuation of innate immune activation. In one embodiment of the invention mTOR inhibitors are administered together with activators of innate immunity in order to augment enhancement of T cell activity. Said T cell activity is augmented for the purpose of increase immunity to tumors, viral, parasitic or bacterial infections.

In one embodiment, mTOR inhibitors are administered with agents known to induce activation of immunity, for example, adjuvant compounds which are known in the art to boost the activity of the immune system. Some of the most commonly studied adjuvants are listed below, but many more are under development. For example, Levamisole, a drug originally used against parasitic infections, has recently been found to improve survival rates among people with colorectal cancer when used together with some chemotherapy drugs [2-8]. It is often used as an immunotherapy adjuvant because it can activate T lymphocytes [9-11]. Additionally, the compound has been demonstrated to induce maturation of dendritic cells, further supporting an immune modulatory role [12]. Levamisole is now used routinely for people with some stages of colorectal cancer and is being tested in clinical trials as a treatment for other types of cancer. Additionally, it has been shown to augment efficacy of other immunotherapeutic agents such as interferon [13, 14]. Aluminum hydroxide (alum) is one of the most common adjuvants used in clinical trials for cancer vaccines. It is already used in vaccines against several infectious agents, including the hepatitis B virus. Bacille Calmette-Guerin (BCG) is a bacterium that is related to the bacterium that causes tuberculosis. The effect of BCG infection on the immune system makes this bacterium useful as a form of anticancer immunotherapy [15]. BCG was one of the earliest immunotherapies used against cancer, either alone, or in combination with other therapies such as hormonal, chemotherapy or radiotherapy [16-24]. It is FDA approved as a routine treatment for superficial bladder cancer. Its usefulness in other cancers as a nonspecific adjuvant is also being tested or has demonstrated therapeutic effects [25-33]. Researchers are looking at injecting BCG to give an added stimuli to the immune system when using chemotherapy, radiation therapy, or other types of immunotherapy. Thus in various embodiments of the current invention, one of skill in the art is directed towards references which have utilized BCG as an adjuvant for other therapies for concentrations and dosing regimens that would apply to the current invention for elicitation of immunity towards proliferating endothelial cells. Incomplete Freund's Adjuvant (IFA) is given together with some experimental therapies to help stimulate the immune system and to increase the immune response to cancer vaccines, both protein and peptide in part by providing a localization factor for T cells [34-42]. IFA is a liquid consisting of an emulsifier in white mineral oil. Another vaccine adjuvant useful for the present invention is interferon alpha, which has been demonstrated to augment NK cell activity, as well as to promote T cell activation and survival [43]. QS-21 is a relatively new immune stimulant made from a plant extract that increases the immune response to vaccines used against melanoma. DETOX is another relatively new adjuvant. It is made from parts of the cell walls of bacteria and a kind of fat. It is used with various immunotherapies to stimulate the immune system. Keyhole limpet hemocyanin (KLH) is another adjuvant used to boost the effectiveness of cancer vaccine therapies. It is extracted from a type of sea mollusc. Dinitrophenyl (DNP) is a hapten/small molecule that can attach to tumor antigens and cause an enhanced immune response. It is used to modify tumor cells in certain cancer vaccines.

Antagonist: As used herein, the term “antagonist” refers to an agent that i) inhibits, decreases or reduces the effects of another agent; and/or ii) inhibits, decreases, reduces, or delays one or more biological events. Antagonists may be or include agents of any chemical class including, for example, small molecules, polypeptides, nucleic acids, carbohydrates, lipids, metals, and/or any other entity that shows the relevant inhibitory activity. An antagonist may be direct (in which case it exerts its influence directly upon its target) or indirect (in which case it exerts its influence by other than binding to its target; e.g., by interacting with a regulator of the target, for example so that level or activity of the target is altered).

Antibody: As is known in the art, an “antibody” is an immunoglobulin that binds specifically to a particular antigen. The term encompasses immunoglobulins that are naturally produced in that they are generated by an organism reacting to the antigen, and also those that are synthetically produced or engineered. An antibody may be monoclonal or polyclonal. An antibody may be a member of any immunoglobulin class, including any of the human classes: IgG, IgM, IgA, and IgD. A typical immunoglobulin (antibody) structural unit as understood in the art, is known to comprise a tetramer. Each tetramer is composed of two identical pairs of polypeptide chains, each pair having one “light” (approximately 25 kD) and one “heavy” chain (approximately 50-70 kD). The N-terminus of each chain defines a variable region of about 100 to 110 or more amino acids primarily responsible for antigen recognition. The terms “variable light chain” (VL) and “variable heavy chain” (VH) refer to these light and heavy chains respectively. Each variable region is further subdivided into hypervariable (HV) and framework (FR) regions. The hypervariable regions comprise three areas of hypervariability sequence called complementarity determining regions (CDR 1, CDR 2 and CDR 3), separated by four framework regions (FR1, FR2, FR2, and FR4) which form a beta-sheet structure and serve as a scaffold to hold the HV regions in position. The C-terminus of each heavy and light chain defines a constant region consisting of one domain for the light chain (CL) and three for the heavy chain (CH1, CH2 and CH3). In some embodiments, the term “full length” is used in reference to an antibody to mean that it contains two heavy chains and two light chains, optionally associated by disulfide bonds as occurs with naturally-produced antibodies. In some embodiments, an antibody is produced by a cell. In some embodiments, an antibody is produced by chemical synthesis. In some embodiments, an antibody is derived from a mammal. In some embodiments, an antibody is derived from an animal such as, but not limited to, mouse, rat, horse, pig, or goat. In some embodiments, an antibody is produced using a recombinant cell culture system. In some embodiments, an antibody may be a purified antibody (for example, by immune-affinity chromatography). In some embodiments, an antibody may be a human antibody. In some embodiments, an antibody may be a humanized antibody (antibody from non-human species whose protein sequences have been modified to increase their similarity to antibody variants produced naturally in humans). In some embodiments, an antibody may be a chimeric antibody (antibody made by combining genetic material from a non-human source, e.g., mouse, rat, horse, or pig, with genetic material from humans).

Antibody agent: As used herein, the term “antibody agent” refers to an agent that specifically binds to a particular antigen. In some embodiments, the term encompasses any polypeptide with immunoglobulin structural elements sufficient to confer specific binding. Suitable antibody agents include, but are not limited to, human antibodies, primatized antibodies, chimeric antibodies, bi-specific antibodies, humanized antibodies, conjugated antibodies (i.e., antibodies conjugated or fused to other proteins, radiolabels, cytotoxins), Small Modular ImmunoPharmaceuticals (“SMIPs™”), single chain antibodies, cameloid antibodies, and antibody fragments. As used herein, the term “antibody agent” also includes intact monoclonal antibodies, polyclonal antibodies, single domain antibodies (e.g., shark single domain antibodies (e.g., IgNAR or fragments thereof)), multispecific antibodies (e.g. bi-specific antibodies) formed from at least two intact antibodies, and antibody fragments so long as they exhibit the desired biological activity. In some embodiments, the term encompasses stapled peptides. In some embodiments, the term encompasses one or more antibody-like binding peptidomimetics. In some embodiments, the term encompasses one or more antibody-like binding scaffold proteins. In come embodiments, the term encompasses monobodies or adnectins. In many embodiments, an antibody agent is or comprises a polypeptide whose amino acid sequence includes one or more structural elements recognized by those skilled in the art as a complementarity determining region (CDR); in some embodiments an antibody agent is or comprises a polypeptide whose amino acid sequence includes at least one CDR (e.g., at least one heavy chain CDR and/or at least one light chain CDR) that is substantially identical to one found in a reference antibody. In some embodiments an included CDR is substantially identical to a reference CDR in that it is either identical in sequence or contains between 1-5 amino acid substitutions as compared with the reference CDR. In some embodiments an included CDR is substantially identical to a reference CDR in that it shows at least 85%, 86%, 87%, 88%, 89%, 90%, 91%, 92%, 93%, 94%, 95%, 96%, 97%, 98%, 99%, or 100% sequence identity with the reference CDR. In some embodiments an included CDR is substantially identical to a reference CDR in that it shows at least 96%, 96%, 97%, 98%, 99%, or 100% sequence identity with the reference CDR. In some embodiments an included CDR is substantially identical to a reference CDR in that at least one amino acid within the included CDR is deleted, added, or substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments an included CDR is substantially identical to a reference CDR in that at least one amino acid within the included CDR is substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an antibody agent is or comprises a polypeptide whose amino acid sequence includes structural elements recognized by those skilled in the art as an immunoglobulin variable domain. In some embodiments, an antibody agent is a polypeptide protein having a binding domain which is homologous or largely homologous to an immunoglobulin-binding domain.

In one embodiment of the invention mTOR inhibitors are administered through controlled release formulations for the purpose of immune modulation, said controlled release means include the use of biocompatible carriers. Such carriers are known in the art and include biodegradable polymers that may be suitable as carriers herein include, without limitation, polycaprolactone, poly(L-lactide), poly(D,L-lactide), poly(D,L-lactide-co-PEG) block copolymers, poly(D,L-lactide-co-trimethylene carbonate), polyglycolide, poly(lactide-co-glycolide), polydioxanone (PDS), polyorthoester, polyanhydride, poly(glycolic acid-co-trimethylene carbonate), polyphosphoester, polyphosphoester urethane, poly(amino acids), polycyanoacrylates, poly(trimethylene carbonate), poly(iminocarbonate), polycarbonates, polyurethanes, copoly(ether-esters) (e.g. PEO/PLA), polyalkylene oxalates, polyphosphazenes, PHA-PEG, and combinations thereof. The PHA may include poly(a-hydroxyacids), poly(.beta.-hydroxyacid) such as poly(3-hydroxybutyrate) (PHB), poly(3-hydroxybutyrate-co-valerate) (PHBV), poly(3-hydroxyproprionate) (PHP), poly(3-hydroxyhexanoate) (PHH), or poly(4-hydroxyacid) such as poly poly(4-hydroxybutyrate), poly(4-hydroxyvalerate), poly(4-hydroxyhexanoate), poly(hydroxyvalerate), poly(tyrosine carbonates), poly(tyrosine arylates), poly(ester amide), polyhydroxyalkanoates (PHA), poly(3-hydroxyalkanoates) such as poly(3-hydroxypropanoate), poly(3-hydroxybutyrate), poly(3-hydroxyvalerate), poly(3-hydroxyhexanoate), poly(3-hydroxyheptanoate) and poly(3-hydroxyoctanoate), poly(4-hydroxyalkanaote) such as poly(4-hydroxybutyrate), poly(4-hydroxyvalerate), poly(4-hydroxyhexanote), poly(4-hydroxyheptanoate), poly(4-hydroxyoctanoate) and copolymers including any of the 3-hydroxyalkanoate or 4-hydroxyalkanoate monomers described herein or blends thereof, polyglycolide, poly(D,L-lactide-co-glycolide), poly(L-lactide-co-glycolide), polycaprolactone, poly(lactide-co-caprolactone), poly(glycolide-co-caprolactone), poly(dioxanone), poly(ortho esters), poly(anhydrides), poly(tyrosine carbonates) and derivatives thereof, poly(tyrosine ester) and derivatives thereof, poly(imino carbonates), poly(glycolic acid-co-trimethylene carbonate), polyphosphoester, polyphosphoester urethane, poly(amino acids), polycyanoacrylates, poly(trimethylene carbonate), poly(iminocarbonate), polyphosphazenes, silicones, polyesters, polyolefins, polyisobutylene and ethylene-alphaolefin copolymers, acrylic polymers and copolymers, vinyl halide polymers and copolymers, such as polyvinyl chloride, polyvinyl ethers, such as polyvinyl methyl ether, polyvinylidene halides, such as polyvinylidene chloride, polyacrylonitrile, polyvinyl ketones, polyvinyl aromatics, such as polystyrene, polyvinyl esters, such as polyvinyl acetate, copolymers of vinyl monomers with each other and olefins, such as ethylene-methyl methacrylate copolymers, acrylonitrile-styrene copolymers, ABS resins, and ethylene-vinyl acetate copolymers, polyamides, such as Nylon 66 and polycaprolactam, alkyd resins, polycarbonates, polyoxymethylenes, polyimides, polyethers, poly(glyceryl sebacate), poly(propylene fumarate), poly(n-butyl methacrylate), poly(sec-butyl methacrylate), poly(isobutyl methacrylate), poly(tert-butyl methacrylate), poly(n-propyl methacrylate), poly(isopropyl methacrylate), poly(ethyl methacrylate), poly(methyl methacrylate), epoxy resins, polyurethanes, rayon, rayon-triacetate, cellulose acetate, cellulose butyrate, cellulose acetate butyrate, cellophane, cellulose nitrate, cellulose propionate, cellulose ethers, carboxymethyl cellulose, polyethers such as poly(ethylene glycol) (PEG), copoly(ether-esters) (e.g. poly(ethylene oxide-co-lactic acid) (PEO/PLA)), polyalkylene oxides such as poly(ethylene oxide), poly(propylene oxide), poly(ether ester), polyalkylene oxalates, phosphoryl choline containing polymer, choline, poly(aspirin), polymers and co-polymers of hydroxyl bearing monomers such as 2-hydroxyethyl methacrylate (HEMA), hydroxypropyl methacrylate (HPMA), hydroxypropylmethacrylamide, PEG acrylate (PEGA), PEG methacrylate, methacrylate polymers containing 2-methacryloyloxyethyl-phosphorylcholine (MPC) and n-vinyl pyrrolidone (VP), carboxylic acid bearing monomers such as methacrylic acid (MA), acrylic acid (AA), alkoxymethacrylate, alkoxyacrylate, and 3-trimethylsilylpropyl methacrylate (TMSPMA), poly(styrene-isoprene-styrene)-PEG (SIS-PEG), polystyrene-PEG, polyisobutylene-PEG, polycaprolactone-PEG (PCL-PEG), PLA-PEG, poly(methyl methacrylate)-PEG (PMMA-PEG), polydimethylsiloxane-co-PEG (PDMS-PEG), poly(vinylidene fluoride)-PEG (PVDF-PEG), PLURONIC™ surfactants (polypropylene oxide-co-polyethylene glycol), poly(tetramethylene glycol), hydroxy functional poly(vinyl pyrrolidone), biomolecules such as collagen, chitosan, alginate, fibrin, fibrinogen, cellulose, starch, dextran, dextrin, hyaluronic acid, fragments and derivatives of hyaluronic acid, heparin, fragments and derivatives of heparin, glycosamino glycan (GAG), GAG derivatives, polysaccharide, elastin, elastin protein mimetics, or combinations thereof.

1. A method of stimulating immune responses in a cancer patient comprising the steps of: a) identifying a patient suffering from cancer possessing a reduced immune response; and b) administering an effective amount of an mTOR inhibitor to augment said immune response.

2. The method of claim 1, wherein said immune response is associated with T cells acquiring antigen specific and/or antigen non-specific proliferative and/or cytokine producing responses and/or cytotoxic responses.

3. The method of claim 1, wherein said immune response is T cell proliferation in response to a combination of Signal 1 and Signal 2.

4. The method of claim 3, wherein said Signal 1 is a T cell receptor generated signal and wherein said Signal 2 is a costimulatory molecule generated signal.

5. The method of claim 4, wherein said costimulatory molecule is selected from the group consisting of: a) CD40; b) CD80; c) CD86; d) 4.1bb; e) OX40; f) IL-2; g)) IL-7; h) IL-11; i) IL-12; j) IL-15; k) IL-17; 1) IL-18 and m) interferon gamma.

6. The method of claim 1, wherein said immune response is reduced as a result of a tumor associated immune suppressive factor.

7. The method of claim 6, wherein said tumor associated immune suppressive factor is selected from the group consisting of: a) PGE-2; b) TGF-beta; c) VEGF; d) IL-10; e) PD-1L; f) arginase catabolites; g) indolamine 2,3 deoxygenase catabolites; h) free adenosine; and i) soluble fas ligand trimers.

8. The method of claim 1, wherein said mTOR inhibitor is selected from the group consisting of: a) everolimus; b) sirolimus; c) temsirolimus; d) dactolisib; e) GSK2126458; f) XL765; g) AZD8055; h) INK128/MLN0128; i) OSI0271 and j) RapaLinks.

9. A method of treating cancer comprising the steps of: a) administering to a cancer patient a checkpoint inhibitor; and b) administering to said cancer patient an mTOR inhibitor.

10. The method of claim 9, wherein said checkpoint inhibitor is an agent blocking inhibitory immune cell signaling.

11. The method of claim 10, wherein said checkpoint inhibitor is Pembrolizumab.

12. The method of claim 10, wherein said checkpoint inhibitor is Nivolumab.

13. The method of claim 10, wherein said checkpoint inhibitor is Atezolizumab.

14. The method of claim 10, wherein said checkpoint inhibitor is Ipilimumab.

15. The method of claim 10, wherein said checkpoint inhibitor blocks an inhibitory receptor selected from the group consisting of: a) a siglec receptor; b) iL-10 receptor; c) IL-13 receptor; d) CTLA-4; e) PD-1; f) PD-1L; and g) TGF-beta receptor

16. The method of claim 9, wherein said mTOR inhibitor is selected from a group comprising of: a) everolimus; b) sirolimus; c) temsirolimus; d) dactolisib; e) GSK2126458; f) XL765; g) AZD8055; h) INK128/MLN0128; i) OSI0271 and j) RapaLinks.

17. The method of claim 9 wherein a stimulator of innate immunity is further added in order to treat said cancer patient.

18. The method of claim 17, wherein said innate immune stimulator induces activation of dendritic cells and/or natural killer cells.

19. The method of claim 18, wherein said innate immune stimulator is Poly IC.

20. The method of claim 18, wherein said innate immune stimulator is beta glucan.
